(19) United States
(12) Patent Application Publication
(10) Pub. No.: US 2014/0100388 A1
(43) Pub. Date: Apr. 10, 2014

(54) FORMIC ACID PRODUCING APPARATUS AND METHOD FOR PRODUCING FORMIC ACID USING THE SAME

(71) Applicant: FORMIC ACID-HYDROGEN ENERGY DEVELOP, Kyoto (JP)

(72) Inventor: Masaru NAKAHARA, Otsu-shi (JP)

(73) Assignee: FORMIC ACID-HYDROGEN ENERGY DEVELOPMENT CORPORATION, Kyoto (JP)

(21) Appl. No.: 13/645,982

(22) Filed: Oct. 5, 2012

(51) Int. Cl. B01J 19/32; C07C 51/15; B01J 10/00

(52) U.S. Cl. 562/609; 422/129; 422/198

(57) ABSTRACT

A formic acid producing apparatus comprising a closed formic acid synthesis reaction section to which an ionic liquid, hydrogen, and carbon dioxide are introduced externally, and in which formic acid is synthesized.

### BACKGROUND OF THE INVENTION

1. Field of the Invention

The present invention relates to a formic acid producing apparatus in which formic acid can be synthesized from hydrogen and carbon dioxide in the presence of an ionic liquid, and to a method for producing formic acid using the apparatus.

2. Description of the Related Art

Formic acid has long been playing an important role as a material for synthesis of various chemical substances such as medicines and agricultural chemicals. Besides, formic acid is important also as a material for production of hydrogen by the hydrogen production method proposed by the present inventor and others through Japanese Patent No. 4481060. There are various known methods for producing formic acid, including a method for producing formic acid from hydrogen and carbon dioxide (see Japanese Unexamined Patent Application Publication No. HEI 7(1995)-173098, for example).

### DISCLOSURE OF THE INVENTION

### Problems to be Solved by the Invention

However, the method disclosed in Japanese Unexamined Patent Application Publication No. HEI 7(1995)-173098, which comprises the reaction of carbon dioxide in a supercritical state (critical point: pressure of 73.3 bar, temperature of 31° C.) with hydrogen in the presence of a metal catalyst (group VIII transition metal complex) and an excess amount of a basic substance, has some limitations such as requirement for the production facility to have high pressure resistance and requirement for separation of the basic substance from the formic acid generated.

### SUMMARY OF THE INVENTION

### Means for Solving the Problems

In view of the abovementioned background, the present inventor has made intensive studies and, as a result, succeeded in developing an apparatus capable of producing formic acid under moderate reaction conditions by simple procedures to complete the present invention.

According to the present invention, therefore, there is provided a formic acid producing apparatus comprising a closed formic acid synthesis reaction section to which an ionic liquid, hydrogen, and carbon dioxide are introduced externally, and in which formic acid is synthesized.

The “ionic liquid” used in the present invention is an organic salt composed of the cation and the anion, which is a viscous liquid in a liquid state in a temperature range between room temperature (15 to 25° C.) and 150° C., having an extremely low vapor pressure and having a viscosity of 20 to 7000 cP at room temperature (1 to 30° C.). The ionic liquid will be described later in detail.

According to the present invention, furthermore, there can be provided a method for producing formic acid comprising synthesizing formic acid from hydrogen and carbon dioxide in the presence of an ionic liquid using the formic acid producing apparatus.

### Effects of the Invention

According to the formic acid producing apparatus due to the present invention, it is possible to produce formic acid in the presence of a repeatedly usable ionic liquid under moderate reaction conditions by simple procedures. Accordingly, the requirement for a reactor to have heat resistance and pressure resistance can be relaxed to reduce device costs, and the consumption of the ionic liquid can be minimized.

### BRIEF DESCRIPTION OF THE DRAWINGS

FIG. 1 is a schematic configuration diagram illustrating Embodiment 1 of a formic acid producing apparatus due to the present invention;

FIG. 2 is a schematic configuration diagram illustrating Embodiment 2 of the formic acid producing apparatus due to the present invention;

FIG. 3A is a side view illustrating a configuration of spreading part in Embodiment 2 of the formic acid producing apparatus;

FIG. 3B is a front view illustrating the configuration of the spreading part in Embodiment 2 of the formic acid producing apparatus;

FIG. 4A is a side view illustrating a configuration of another spreading part in Embodiment 2 of the formic acid producing apparatus;

FIG. 4B is a front view illustrating the configuration of the spreading part in Embodiment 2 of the formic acid producing apparatus;

FIG. 5 is a schematic perspective view illustrating a formic acid synthesis reaction section in Embodiment 3 of the formic acid producing apparatus due to the present invention (top panel not shown);

FIGS. 6A and 6B are graphs showing ¹H NMR (Nuclear magnetic resonance) spectra obtained before and after a reaction in Synthesis Example 4; and

FIG. 7 is a graph showing formic acid yields in Synthesis Examples 1 to 5 (reaction temperature: 60 to 140° C.).

### DESCRIPTION OF THE PREFERRED EMBODIMENTS

### <Formic Acid Producing Apparatus>

A formic acid producing apparatus due to the present invention comprises a closed formic acid synthesis reaction section to which an ionic liquid, hydrogen, and carbon dioxide are introduced externally, and in which formic acid is synthesized.

In the formic acid producing apparatus due to the present invention, parts that contact with formic acid are preferably formed of a material having corrosion resistance. Examples of the material having corrosion resistance against formic acid include ceramics; glass; metals such as Ti—Pd alloys, pure zirconium, Ni—Mo—Cr alloys, and stainless steel; thermoplastic resins such as polypropylene (PP), polytetrafluoroethylene (PTFE), polyethylene (PE), rigid polyvinyl chloride (PVC), polysulfone (PSF), polyvinylidene chloride (PVDC), polyvinyl alcohol (PVA), fluoro plastics (PTFE), and methylpentene resin (TPX); thermosetting resins such as phenol resins (PF) and furan resins (FF); and composite materials obtained by coating surfaces of the abovementioned metals with ceramics, glass or the abovementioned resins. It is not preferable to use thermoplastic resins, because the ionic liquid is to be heated.

In the present invention, the formic acid synthesis reaction section must include a closed reactor and may have the following configurations (1) to (4) to achieve efficient formic acid synthesis. The efficiency can be increased when the configurations (1) to (4) are combined.

(1) The formic acid synthesis reaction section preferably further comprises a closed reactor having an outlet for sending out formic acid synthesized to an exterior; a heating means for heating at least the ionic liquid in the reactor; and a spreading part provided in the reactor for expanding the surface area of the ionic liquid in the reactor.

The spreading part is a synthesis reaction accelerating means for increasing efficiency of synthesis of formic acid from hydrogen and carbon dioxide by expanding the surface area of the ionic liquid in the reactor and thereby expanding (increasing) areas where a mixture of hydrogen and carbon dioxide contacts with the ionic liquid. Thus, the areas where the mixture of hydrogen and carbon dioxide contacts with the ionic liquid are expanded to provide more interfaces for the mixture to contact with the ionic liquid. Further, since a chemical reaction is accelerated by a locally intensified electric field gradient generated at an interface between the mixture and the ionic liquid, the spreading part is also aimed at creating more interfaces to accelerate the formic acid synthesis reaction. According to the configuration, the mixture does not need to be at a high temperature and in a high energy state, because the formic acid synthesis reaction progresses even at a low temperature due to the local electric field gradient generated at such interfaces.

(2) The spreading part preferably has a spreading wall; and a driving means for moving the spreading wall.

According to the configuration (2), it is possible to easily expand the surface area of the ionic liquid in a limited space in the reactor. Since ionic liquids are generally viscous, and therefore the areas where the mixture of hydrogen and carbon dioxide contacts with the ionic liquid are not easily increased even if the ionic liquid in the reactor is vigorously stirred, it is difficult to synthesize formic acid efficiently. Against the problem, the spreading wall is the most appropriate means for expanding the surface area of the viscous ionic liquid and accelerating the formic acid synthesis reaction. In particular, a technique of rotating the spreading wall in the reactor is effective for expanding the surface area of a highly viscous ionic liquid with a simple mechanism and less drive energy.

Though not particularly limited, the shape and the movement of the movable spreading wall are preferably those that allow significant expansion of the surface area of the ionic liquid in a small space. For example, one or more (preferably a plurality of) discoid spreading walls may be connected in parallel in a skewered state around a rotation axis and disposed so that a lower part of each spreading wall is immersed in the ionic liquid, and the rotation axis may be rotated by a driving means such as a motor. According to the configuration, it is possible to rotate the spreading walls with relatively low resistance against the highly viscous ionic liquid to raise the ionic liquid adhering to the spreading walls above the liquid level and cause the ionic liquid to run along surfaces of the spreading walls successively.

Other than rotation, the movement of the movable spreading wall may be pendular-like swing, seesaw-like turn, up-and-down reciprocating motion, and the like, which also can expand the surface area of the ionic liquid.

(3) The spreading part preferably comprises a spreading wall; and a liquid circulation line for drawing the ionic liquid in the reactor and discharging the ionic liquid toward the spreading wall.

In this case, the spreading wall is fixed unlike the movable spreading wall. For example, one or more spreading walls are provided in the reactor to divide the internal space of the reactor into a plurality of compartments, so that the ionic liquid runs downward along both side surfaces of each spreading wall.

The configuration (3) is advantageous, because the surface area of the ionic liquid can be easily expanded in a limited space in the reactor, and besides the reactor can be reinforced. In addition, it is preferable to use inner surfaces of enclosing walls defining the outline of the reactor as the side surfaces of the spreading walls, because in this case, the surface area of the ionic liquid can be further expanded. This configuration is suitable for the case where an ionic liquid having relatively low viscosity is used.

The liquid circulation line is useful in repeated use of the ionic liquid and is able to circulate a highly viscous ionic liquid depending on the kind of pump provided therein (for example, plunger pump).

(4) The spreading wall has rough surfaces.

According to the configuration (4), the surface area of the side surfaces of the spreading wall increases to increase the surface area of the ionic liquid running along the side surfaces (rough surfaces) of the spreading wall. As a result, more interfaces between the ionic liquid and the mixture of hydrogen and carbon dioxide are provided to further improve the formic acid synthesis efficiency.

In this case, the rough surfaces can be formed by providing projections, recesses or through holes in the surfaces of the spreading wall. A spreading wall made of a ceramic material is advantageous, because it has naturally formed rough surfaces. The shape and the size of the projections, the recesses and the through holes are not particularly limited, and they may be designed so as to achieve the most efficient formic acid synthesis according to the viscosity of the ionic liquid, the surface area of the spreading wall, and the moving speed of the movable spreading wall, for example.

### <Method for Producing Formic Acid Using Formic Acid Producing Apparatus>

The “ionic liquid” usable in the present invention means, but is not limited to, an ionic liquid of a salt containing a combination of: a cation such as ammonium ions including imidazolium salts and pyridinium salts, and phosphonium ions, which are phosphorus compounds; and an anion such as halogen ions including bromide ion, halogenated alkyloxy ions such as triflate and trifluoroacetoxy ions, boron ions such as tetraphenyl borate, phosphate ions such as hexafluorophosphate.

In terms of repeated use, however, compounds represented by the following general formula (1) are suitable for the ionic liquid. That is, an ionic liquid preferably contains a phosphonium ion as a cation and a hydrophilic anion such as formate ion, chloride ion, or nitrate ion as an anion.

Such compounds allow efficient synthesize of formic acid from hydrogen and carbon dioxide. They are preferable also in terms of availability and ease of molecular design.

In this case, the ionic liquid usable in the present invention can be obtained by using, as a raw material, an ionic liquid having an anion that can be replaced with a formate ion, a chloride ion or a nitrate ion as the anion in the ionic liquid, and replacing the anion in the ionic liquid as a raw material with a formate ion, a chloride ion or a nitrate ion according to the method described in Biomacromolecules, Vol. 7, Pages 3295-3297, 2006. Alternatively, the ionic liquid containing a phosphonium ion and a formate ion, a chloride ion or a nitrate ion may contain the anion in the raw material.

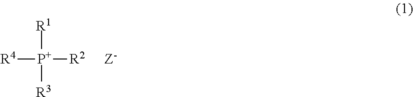

wherein

R¹, R², R³, and R⁴, the same or different, each represent an alkyl group that may be substituted with a halogen atom, or an aryl group or a heteroaryl group that may be substituted at the o-position or the p-position with 1 to 3 halogen atoms or a lower alkyl or alkoxy group, and

Z⁻ represents a counter anion for a phosphonium cation.

In the phosphonium-based ionic liquid represented by the general formula (1), the alkyl group represented by R¹, R², R³, and R⁴ that may be substituted with a halogen atom may be, for example, a linear or branched alkyl group or perfluoroalkyl group having 1 to 18 carbon atoms. Specific examples thereof include methyl, ethyl, n-propyl, iso-propyl, n-butyl, iso-butyl, sec-butyl, tert-butyl, n-pentyl, iso-pentyl, n-hexyl, n-octyl, n-decyl, n-dodecyl, n-tetradecyl, n-octadecyl, trifluoromethyl, pentafluoromethyl, pentafluoroethyl, heptafluoro-n-propyl, heptafluoro-iso-propyl, and nonafluoro-n-butyl groups.

Examples of the aryl group that may be substituted at the o-position or the p-position with 1 to 3 halogen atoms or a lower alkyl or alkoxy group include phenyl and benzyl groups.

Examples of the heteroaryl group that may be substituted at the o-position or the p-position with 1 to 3 halogen atoms or a lower alkyl or alkoxy group include furan and thiophene.

In the phosphonium-based ionic liquid represented by the general formula (1), the phosphonium cation is preferably a phosphonium cation in which R¹, R², and R³ are an alkyl, aryl or hydroxymethyl group having 1 to 18 carbon atoms, and R⁴ is an alkyl, aryl, hydroxymethyl, or cyanomethyl group having 1 to 18 carbon atoms. Particularly preferably, the phosphonium cation is a tetraphenylphosphonium salt, a trihexyltetradecylphosphonium salt, or a tetrabutylphosphonium salt.

Examples of the counter anion for a phosphonium cation represented by Z⁻ include nitride ion (NO₃⁻), p-toluenesulfonate anion, methanesulfonate anion (CH₃SO₃⁻), trifluoromethanesulfonate anion (CF₃SO₃⁻), bis(trifluoromethanesulfonyl)imide anion ((CF₃SO₂)₂N⁻), and formate anion (HCO₂⁻) as well as halide ions such as chloride ion (Cl⁻), bromide ion (Br⁻), and iodide ion (I⁻).

Preferred examples of the ionic liquid include a chloride ion which generates the strongest electric field and an ionic liquid in which a counter anion is a formate anion (i.e., formic acid salt). These liquids are, as media for the production of formic acid by using hydrogen and carbon dioxide as raw materials, excellent in reaction selectivity (high-purity formic acid is produced) and reaction velocity.

The ionic liquid in which a counter anion is a formate anion can be synthesized, for example, from an ionic liquid in which a counter anion is different from a formate anion such as bromide ion, by an anion exchange method with the use of a strong-basic ion exchange resin (Biomacromolecules, Vol. 7, Pages 3295-3297, 2006).

Various kinds of ionic liquids in which a counter anion is different from a formate anion are commercially available. If not commercially available, such ionic liquids can be synthesized, for example, in accordance with the method described in Ionic Liquids in Synthesis I, Wiley-VCH, 2007.

Formic acid is produced from hydrogen and carbon dioxide in the presence of the ionic liquid by bringing the mixture of hydrogen and carbon dioxide into contact with a surface of the ionic liquid in a pressure-resistant reactor. At this time, it is preferable to increase the contact area between the mixture and the ionic liquid by expanding the surface area of the ionic liquid by means of the spreading part.

As carbon dioxide, liquefied carbon dioxide and dry ice may be gasified in the reactor.

Hydrogen and carbon dioxide in the reactor may have a pressure of 5 to 1000 bar. Specifically, the pressure is preferably 10 to 500 bar.

The reaction temperature is not particularly limited as long as the temperature allows the ionic liquid to stay in a liquid state, and it is preferable to raise the temperature to room temperature or more in order to produce formic acid efficiently. In this case, the upper limit of the reaction temperature is 250° C. and preferably 60 to 140° C., for example, because a too high temperature may cause degradation of the ionic liquid and reduce the amount of formic acid to be generated due to a thermodynamic reason. The reaction time is 1 hour to 100 hours, for example.

While being capable of producing formic acid simply and easily without the use of a metal catalyst, the formic acid production method due to the present invention may use a metal catalyst.

By using a metal catalyst, formic acid can be generated at a high rate even when the heating temperature is 50° C. or more lower than the heating temperature preferred in the case where no metal catalyst is used (even when the heating temperature is 50° C., for example).

Examples of the metal catalyst include simple salts (chlorides and oxides) and complexes (specific examples of ligands: amines, phosphines, conjugated dienes, and the like) of transition metals (Ru, Rh, Ir, and the like) in groups 8, 9, and 10 of the periodic table. More specifically, dichlorotetrakis(triphenylphosphine)ruthenium can be used as the metal catalyst. In this case, the reaction is accelerated, and therefore the formic acid synthesis proceeds quickly even at a low temperature.

The amount of the metal catalyst to use may be, for example, approximately 0.01 to 3% of the total weight of the ionic liquid. Specifically, 0.01 to 3 parts by weight of a metal catalyst with respect to 100 parts by weight of the ionic liquid in the formic acid synthesis reaction section is added to the ionic liquid or carried by a carrier such as, for example, a porous ceramic and the metal catalyst carrier is fixed in the reactor in the formic acid synthesis reaction section.

Hereinafter, embodiments of the formic acid producing apparatus due to the present invention will be described in detail with reference to the drawings. In the following embodiments, hydrogen and carbon dioxide are in a gaseous state by way of example, but they do not need to be limited to the three states of matter.

### Embodiment 1

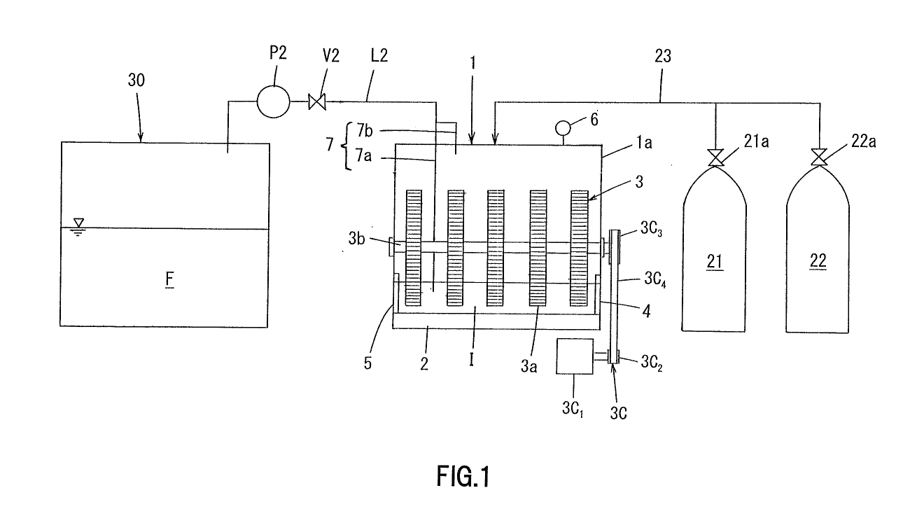

FIG. 1 is a schematic configuration diagram illustrating Embodiment 1 of a formic acid producing apparatus due to the present invention.

First, the configuration of a formic acid producing apparatus according to Embodiment 1 will be described, and then the operation of the formic acid producing apparatus will be described.

As illustrated in FIG. 1, the formic acid producing apparatus according to Embodiment 1 due to the present invention includes a formic acid synthesis reaction section 1.

Specifically, the formic acid synthesis reaction section 1 includes a closable reactor 1a for containing an ionic liquid I, a heater 2 for heating the ionic liquid I, a spreading part 3 for expanding the surface area of the ionic liquid I, a temperature sensor 4 for detecting the temperature of the ionic liquid I and a level sensor 5 for detecting the liquid level of the ionic liquid I. The formic acid synthesis reaction section 1 of Embodiment 1 is suitable for the case where the ionic liquid I to use has high viscosity, for example. In particular, when the ionic liquid has high viscosity and formic acid has low solubility, formic acid synthesized is likely to stay on the ionic liquid I, and therefore the level sensor 5 will detect the liquid level of formic acid on the ionic liquid I. When the ionic liquid has low viscosity and formic acid has high solubility, they are separated by causing phase separation by cooling and extracting the formic acid from the ionic liquid, and then the formic acid is detected by a concentration sensor, for example.

The reactor 1a is a pressure-resistant container formed into a rectangular parallelepiped for containing the ionic liquid I. The reactor 1a is formed of an inner vessel made of a ceramic material and an outer vessel made of stainless steel, for example. In addition, the reactor 1a has a manometer 6 for determining the inner pressure of the reactor and an outlet section 7 for sending out the formic acid synthesized in the reactor to an exterior. The reactor 1a may be covered with a heat insulating material to reduce escape of heat in the reactor 1a.

The reactor 1a is connected to a hydrogen cylinder 21 as a source of hydrogen and a carbon dioxide cylinder 22 as a source of carbon dioxide through a gas supply pipe 23, and hydrogen gas and carbon dioxide gas as raw materials of formic acid are supplied into the reactor 1a at a predetermined pressure ratio. The hydrogen cylinder 21 and the carbon dioxide cylinder 22 have pressure regulating valves 21a and 22a, respectively.

The outlet section 7 is configured to have, for example, a long delivery pipe 7a and a short delivery pipe 7b that are penetrating an upper wall of the reactor 1a so as to send out a formic acid solution or formic acid gas in the reactor 1a to an exterior. The short delivery pipe 7b may be provided with a shut-off valve.

Specifically, the long delivery pipe 7a is disposed so that a lower end thereof opens in the formic acid solution staying on the ionic liquid I in the reactor 1a. On the other hand, the short delivery pipe 7b is disposed above the liquid level of the formic acid solution staying on the ionic liquid I in the reactor 1a. The long delivery pipe 7a and the short delivery pipe 7b have ends projecting out of the reactor 1a, each of which is connected to an upstream end of a pipe line L2 having a pump P2 and a valve V2. A downstream end of the pipe line L2 is connected to a formic acid tank 30 for storing formic acid F.

The spreading part 3 is provided in the reactor 1a and expands the surface area of the ionic liquid I in the reactor 1a. In the case of Embodiment 1, the spreading part 3 includes a plurality of discoid spreading walls 3a, a rotation axis 3b for connecting the spreading walls in parallel in a skewered state, and a driving means 3c for rotating the rotation axis. The spreading part 3 is disposed so that a lower part of each spreading wall 3a is immersed in the ionic liquid I. The spreading walls 3a are each made of a discoid ceramic having a central hole through which the rotation axis 3b is inserted, and each have a peripheral surface having a plurality of grooves, for example. The spreading walls 3a are not limited to the abovementioned shape and may be in the shape of a gear.

The rotation axis 3b is also made of a ceramic material. The spreading walls 3a and the rotation axis 3b are coupled by cutout recesses and engagement projections formed in the central holes of the spreading walls 3a and the peripheral surface of the rotation axis 3b to engage with one another, for example. This configuration allows the spreading walls 3a and the rotation axis 3b to rotate integrally. In order to prevent displacement of the spreading walls 3a, ceramic screws are fixated to the rotation axis 3b at opposite sides of each of the spreading walls 3a.

Both the ends of the rotation axis 3b are attached to opposite walls of the reactor 1a in a rotatable and air-tight manner, and one of the ends of the rotation axis 3b is projected from the reactor 1a to be connected to the driving means 3c.

The driving means 3c includes, for example, a motor 3c₁, a first pulley 3c₂ fixed to an end of a driving shaft of the motor 3c₁, a second pulley 3c₃ fixed to the end of the rotation axis 3b, and a belt 3c₄ laid across the first and second pulleys 3c₂ and 3c₃. Instead of the first and second pulleys 3c₂ and 3c₃, and the belt 3c₄, a sprocket and a chain may be used.

### <Operation of Formic Acid Producing Apparatus>

The formic acid producing apparatus due to the present invention is operated as follows, for example (see FIG. 1).

An operation switch of the formic acid producing apparatus is turned on, and in response, the heater 2 generates heat to heat the ionic liquid I via the reactor 1a. In the meantime, the motor 3c₁ is rotated, and the rotative force of the motor 3c₁ is transmitted to the rotation axis 3b via the first pulley 3c₂, the belt 3c₄, and the second pulley 3c₃ to rotate the rotation axis 3b and the spreading walls 3a.

The rotation of the spreading walls 3a raises the ionic liquid I heated around the spreading walls 3a above the liquid level. When having high viscosity like starch syrup, in particular, the ionic liquid I is highly adhesive to the spreading walls 3a, and therefore raised high above the liquid level as the spreading walls 3a rotate. In addition, the plurality of grooves in the peripheral surfaces of the spreading walls 3a are effective for the raise of the ionic liquid I. Having a discoid shape with a small thickness, on the other hand, the spreading walls 3a can have less rotational resistance to the ionic liquid I and prevent the motor 3c₁ from being overloaded.

In the formic acid synthesis in the formic acid synthesis reaction section 1, hydrogen and carbon dioxide are reacted in the presence of the ionic liquid I by introducing hydrogen and carbon dioxide in the reactor 1a, and bringing a mixed gas of hydrogen and carbon dioxide into contact with a surface of the ionic liquid I. At this time, the surface area of the ionic liquid I is expanded by the rotation of each spreading wall 3a in the spreading part 3 to increase the area where the mixed gas contacts with the ionic liquid I. At this time, the pressure of hydrogen in the reactor 1a is set at, for example, 50 bar, and the pressure of carbon dioxide is set at, for example, 50 bar. The reaction temperature is not particularly limited as long as the temperature allows the ionic liquid I to stay in a liquid state, and it is preferable to raise the temperature to room temperature or more, for example, to 130° C. in order to produce formic acid efficiently. The reaction time is 12 hours, for example.

While the spreading part 3 is in operation, the temperature of the ionic liquid I is detected by the temperature sensor 4, and detection signals thereof are input into a control section, not shown. For example, when the temperature of the ionic liquid I is set to be raised to 150° C., the control section controls the heater 2 so that the heater is turned off when the temperature of the ionic liquid I exceeds 155° C. and turned on when the temperature falls below 145° C., for example.

When the formic acid (formic acid solution) synthesized as described above is accumulated on the ionic liquid I in the reactor 1a, the valve V2 may be opened, and the pump P2 may be driven to send the formic acid solution out of the reactor 1a to the formic acid tank 30. Alternatively, the formic acid may be distilled. That is, the formic acid solution may be heated with the ionic liquid I by the heater 2 in the formic acid synthesis reaction section 1 to generate formic acid gas, and the formic acid gas may be liquefied while entering the pipe line L2 to be sent to the formic acid tank 30.

Instead of the gas cylinders, hydrogen and carbon dioxide emitted from an industrial plant may be directly supplied to the formic acid synthesis reaction section 1. In this case, hydrogen and carbon dioxide cannot be supplied to the formic acid synthesis reaction section 1 during the synthesis and the delivery of formic acid, and it is therefore preferable to provide a plurality of formic acid synthesis reaction sections 1 so that hydrogen and carbon dioxide can be supplied to any of the plurality of formic acid synthesis reaction sections 1.

### Embodiment 2

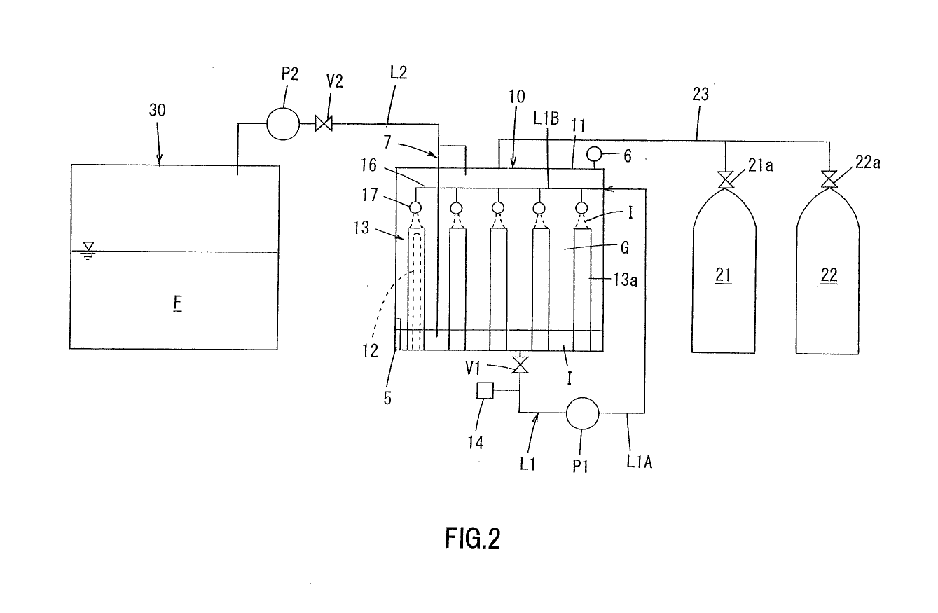

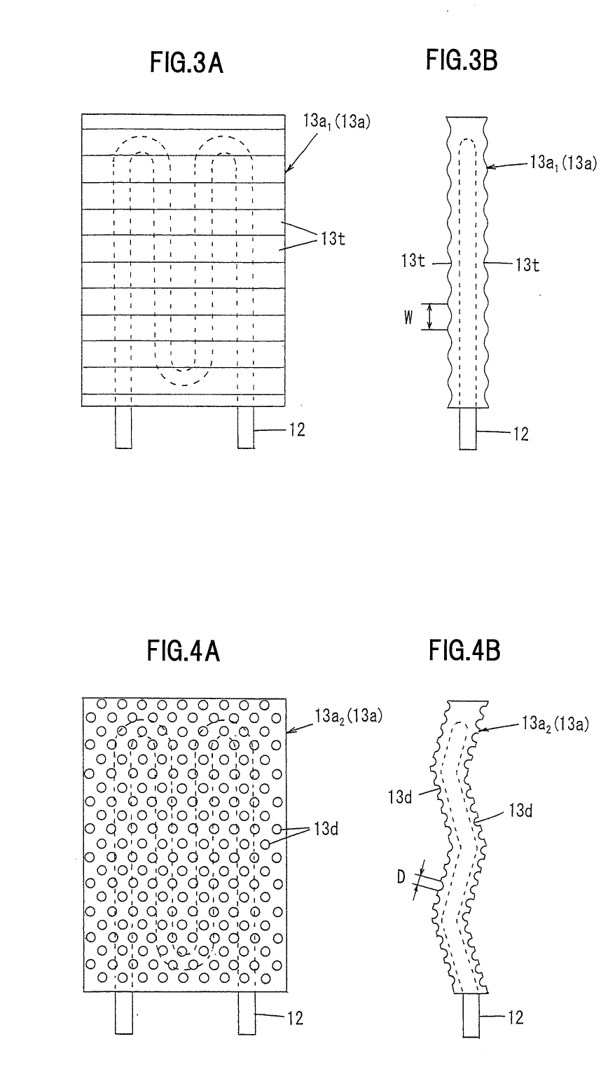

FIG. 2 is a schematic configuration diagram illustrating Embodiment 2 of the formic acid producing apparatus due to the present invention. FIG. 3A is a side view illustrating a configuration of a spreading part in Embodiment 2 of the formic acid producing apparatus, and FIG. 3B is a front view illustrating the same. FIG. 4A is a side view illustrating a configuration of another spreading part in Embodiment 2 of the formic acid producing apparatus, and FIG. 4B is a front view illustrating the same. In FIG. 2, the same components as those in FIG. 1 are represented by the same reference numerals.

Embodiment 2 of the formic acid producing apparatus has the same configurations as Embodiment 1 except for the configuration of the formic acid synthesis reaction section. Hereinafter, differences of Embodiment 2 from Embodiment 1 will be described mainly.

Specifically, a formic acid synthesis reaction section 10 includes a closable reactor 11, heaters 12, a spreading part 13, and a liquid circulation line L1.

The reactor 11 is similar to the reactor 1a of Embodiment 1, having the outlet section 7 for sending out formic acid generated therein to an exterior.

In the case of Embodiment 2, the spreading part 13 has a plurality of spreading walls 13a stood in parallel in the reactor 11.

As illustrated in FIGS. 3 and 4, both the right and left side surfaces of each spreading wall 13a are thoroughly rough thereby to increase the surface area of the spreading wall 13a. In the case of FIGS. 3A and 3B, the whole areas of both the side surfaces of a spreading wall 13a₁ are provided with a plurality of grooves 13t having a width W of, for example, approximately 1 to 10 mm and extending in the horizontal direction into a stripe state to form the rough surfaces. Instead of the grooves 13t, projections may be provided. In the case of FIGS. 4A and 4B, both the side surfaces of a spreading wall 13a₂ bent into a shallow W shape are provided with a plurality of hemispheric recesses 13d having a diameter D of, for example, approximately 1 to 10 mm to form the rough surfaces. Instead of the recesses 13d, projections may be provided.

The heaters 12 heat the ionic liquid I in the reactor 11. In the case of Embodiment 2, the heaters 12 are M-shaped sheathed heaters, provided in the respective spreading walls 13a.

In this case, each spreading wall 13a having a hollow and flat shape with an opening at a lower part is formed of a ceramic material, for example, and one heater 12 is inserted in each spreading wall 13a through the opening. Furthermore, in order to prevent the heaters 12 from corrosion with formic acid, a filler such as, for example, a paste prepared by mixing sand and a thermosetting resin is poured into the spreading wall 13a and thermally cured. Thereby, the heater 12 can be fixed in the spreading wall 13a and prevented from corrosion due to contact with formic acid. In addition, the reactor 11 is provided with through holes in the bottom wall thereof at positions in which each spreading wall 13a is attached. In the respective through holes, both the ends of each heater 12 are inserted, and both the ends of the heater 12 and the bottom wall are welded and fixed so as to prevent liquid leak.

The liquid circulation line L1 draws the ionic liquid I staying at the bottom of the reactor 11 and discharges the ionic liquid I to an upper part of each spreading wall 13a. In the case of Embodiment 2, the liquid circulation line L1 includes a circulation pipe L1A and a discharge section L113 provided to an upper part in the reactor 11.

The circulation pipe L1A is provided with a valve V1, a temperature sensor 14 for detecting the temperature of the ionic liquid I, and a pump P1 on its way. The pump P1 is not particularly limited, and a corrosion-resistant rotary pump is preferable when the ionic liquid is highly viscous. The circulation pipe L1A may be provided with a valved discharge pipe, not shown, for discharging old ionic liquid out of the reactor 11 at a downstream side relative to the pump P1.

The discharge section L1B includes a center pipe 116 connected to a downstream end of the circulation pipe L1A and a plurality of branch pipes 17 diverging from the center pipe 16 and arranged in parallel above an upper end of each spreading wall 13a. The branch pipes 17 each have a plurality of discharge holes along the longer direction for discharging the ionic liquid I.

When the formic acid synthesis reaction section 10 is in operation, each heater 12 generates heat to heat each spreading wall 13a. In the meantime, the valve V1 is opened and the pump P1 is driven, so that the ionic liquid I in the reactor 11 is drawn into the liquid circulation line L1, and the ionic liquid I is discharged from each branch pipe 17 in the discharge section L1B to the upper end of each spreading wall 13a. Thereby, the ionic liquid I runs downward along the right and left side surfaces (rough surfaces) of each spreading wall 13a heated, and the ionic liquid I accumulated at the bottom of the reactor 11 is drawn into the liquid circulation line L1 to reach the upper part of the reactor 11 again and discharged to the upper end of each spreading wall 13a as described above. Thus, the ionic liquid I is continuously circulated in the formic acid synthesis reaction section 10 to be heated by each heater 12 to a temperature range of normal temperature to 200° C. while running along the right and left rough surfaces of each spreading wall 13a.

According to Embodiment 2, it is possible to directly heat the side surfaces of the spreading walls 13a where the formic acid generation efficiency is the highest in the formic acid synthesis reaction section 10, and therefore the formic acid generation efficiency is more improved, and the thermal efficiency is also improved. The heaters 12 may be provided not only in the spreading walls 13a but at the bottom of the reactor 11 and in the liquid circulation line L1, for example.

### Embodiment 3

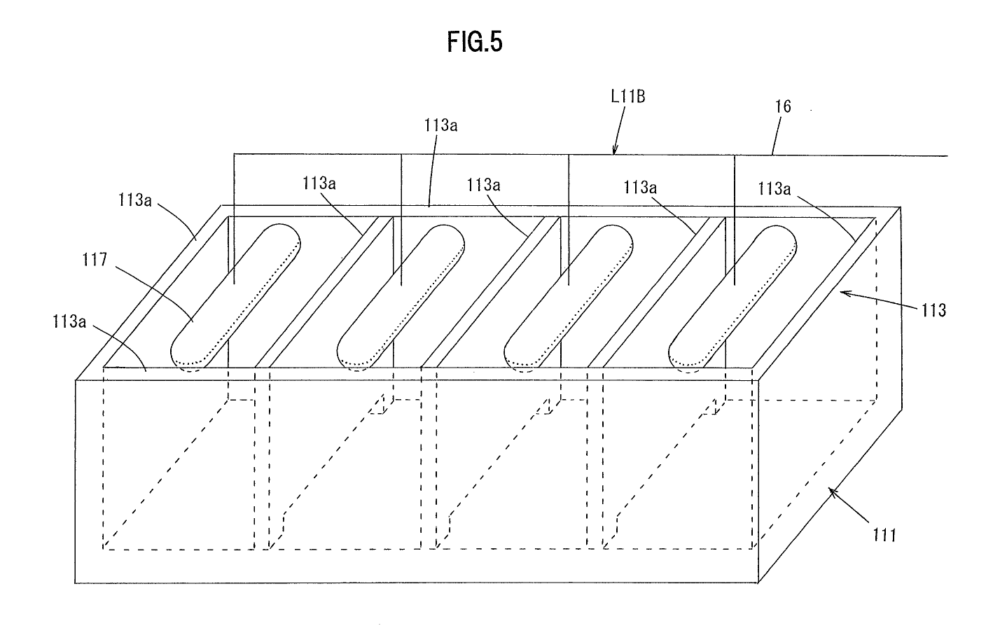

FIG. 5 is a schematic perspective view illustrating a formic acid synthesis reaction section in Embodiment 3 of the formic acid producing apparatus due to the present invention (top panel not shown). In FIG. 5, the same components as those in FIG. 2 are represented by the same reference numerals, and description thereof will be omitted.

Embodiment 3 has the same configurations as Embodiment 2 except for the configuration of a formic acid synthesis reaction section 110. Hereinafter, differences of Embodiment 3 from Embodiment 2 will be described mainly. In Embodiment 3 of the formic acid producing apparatus, the formic acid synthesis reaction section 110 includes a closable reactor 111, heaters, not shown, a spreading part 113 and a liquid circulation line L11 (see FIG. 2). In this case, spreading walls 113a in the spreading part 113 form enclosing walls and partitions of the reactor 111, and the heaters are provided in the respective spreading walls 113a. Preferably, inner surfaces of the spreading walls 113a forming the enclosing walls of the reactor 111 and both surfaces of each spreading wall 113a forming a partition are rough surfaces (see FIGS. 3 and 4).

In the case of Embodiment 3, the liquid circulation line L11 includes a circulation pipe L11A and a discharge section L11B, and the discharge section L11B includes one center pipe 16 connected to a downstream end of the circulation pipe L11A and a plurality of branch pipes 117 diverging from the center pipe 16 and arranged in parallel near a side surface of an upper end of each spreading wall 113a. The branch pipes 117 each have a plurality of discharge holes along the longer direction and the shorter direction for spraying or atomizing the ionic liquid I. That is, the discharge sections L11B in Embodiment 3 are suitable for the case where an ionic liquid having low viscosity is circulated.

In the formic acid synthesis reaction section 110 of Embodiment 3, the ionic liquid I in the form of droplets or mist is released from the branch pipes 117 into the reactor 111 to adhere to upper parts of the side surfaces of the respective spreading walls 113a. The ionic liquid I that has adhered to the upper parts of the side surfaces the respective spreading walls 113a are then heated while running along the side surfaces, and the ionic liquid I heated contacts with the mixed gas of hydrogen and carbon dioxide.

According to Embodiment 3, the ionic liquid I has an increased surface area by running along the side surfaces of the spreading walls 113a, and besides the ionic liquid I has a more increased surface area by being released in the form of droplets or mist from the branch pipes 117. As a result, formic acid can be generated with higher efficiency. In addition, since the enclosing walls constituting the reactor 111 are used as the spreading walls 113a, the number of partitions can be reduced to downsize the reactor 111.

The following synthesis examples further illustrate the present invention, but do not limit the present invention.

### Ionic Liquid

As an ionic liquid to use in the present invention, tri-n-hexyl-n-tetradecylphosphonium chloride (purity: 99%) supplied by Koei Chemical Co., Ltd. was used in Synthesis Examples 1 to 5.

Prior to use, the ionic liquid was heated (120° C.) and dried under vacuum for 12 hours after acquisition. The ionic liquid was studied for the reaction temperature suitable for the formic acid synthesis reaction by the following method.

### Sample Preparation

In an uncatalyzed formic acid synthesis in the ionic liquid, the ionic liquid, hydrogen, and carbon dioxide were put in a silica tube whose surface had no catalyst effect, in order to observe variation with time of hydrogen, carbon dioxide and formic acid by ¹H- and ¹³C-NMR. Since some metals have catalyst effect, the silica tube found in previous studies to have no catalyst effect and allow the NMR observation was used.

A gas phase and a liquid phase were measured by the NMR without opening the silica tube, and the reaction was monitored, focusing on the reversibility of a new version of water gas shift reaction represented by the following formula involving formic acid as an intermediate unlike previously known water gas shift reactions.

(CO+H₂O⇄HCOOH⇄H₂+CO₂)

According to the following method, an aqueous solution of formic acid and the ionic liquid were enclosed in a silica tube for decomposition of formic acid into hydrogen and carbon dioxide thereby to enclose desired amounts of hydrogen, carbon dioxide, and the ionic liquid in the silica tube.

In a dry box filled up with nitrogen gas, 0.29 ml of a mixture of a commercially available aqueous solution of formic acid (purity: 95%) manufactured by Nacalai Tesque, Inc, Japan and the ionic liquid previously dried (mole ratio of formic acid to ionic liquid=4:1) were fed into a silica tube (0.49 mL in capacity) having a size of 10 cm in length×2.5 mm in inner diameter, and the silica tube was sealed by using a gas burner to prepare a sample.

### Synthesis Example 1

A mixture of formic acid and the ionic liquid in an amount of 0.29 mL (mol concentration: 6.88 M) was fed into a silica tube, which was then sealed, to cause the decomposition of the formic acid into hydrogen and carbon dioxide as described above. Thereafter, hydrogen and carbon dioxide were reacted for 12 hours in an electric furnace maintained at 60° C. Specifically, the synthesis experiment was carried out under conditions where the formic acid was decomposed 53% and 3.23 M of formic acid was present in the ionic liquid. After the reaction, the NMR observation was carried out to study the formic acid production.

The formic acid production was determined under the specified conditions on the assumption that the theoretically maximum amount of formic acid from hydrogen and carbon dioxide enclosed is 100% (mol concentration of formic acid dissolved in the ionic liquid: 6.88 M).

The formic acid production refers to the amount of formic acid purely synthesized from hydrogen gas and carbon dioxide gas, excluding the formic acid present at the initiation of the reaction. As a result, the formic acid yield (formic acid production) in the presence of formic acid was found to be 28% (mol concentration: 1.91 M).

### Synthesis Example 2

Formic acid was synthesized in the same manner as in Synthesis Example 1 except that the reaction temperature in Synthesis Example 1 was changed to 80° C.

As a result, the formic acid yield (formic acid production) was found to be 29% (mol concentration: 2.00 M).

### Synthesis Example 3

Formic acid was synthesized in the same manner as in Synthesis Example 1 except that the reaction temperature in Synthesis Example 1 was changed to 100° C.

As a result, the formic acid yield (formic acid production) was found to be 18% (mol concentration: 1.24 M).

### Synthesis Example 4

Formic acid was synthesized in the same manner as in Synthesis Example 1 except that the reaction temperature in Synthesis Example 1 was changed to 130° C.

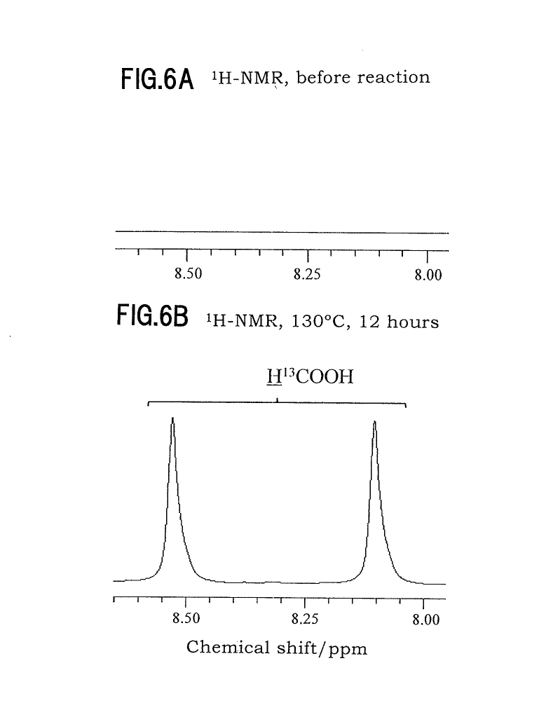

As a result, the formic acid yield (formic acid production) was found to be 28% (mol concentration: 1.96 M). As an example, FIG. 6 shows ¹H-NMR spectral change between before and after the reaction.

### Synthesis Example 5

Formic acid was synthesized in the same manner as in Synthesis Example 1 except that the reaction temperature in Synthesis Example 1 was changed to 140° C.

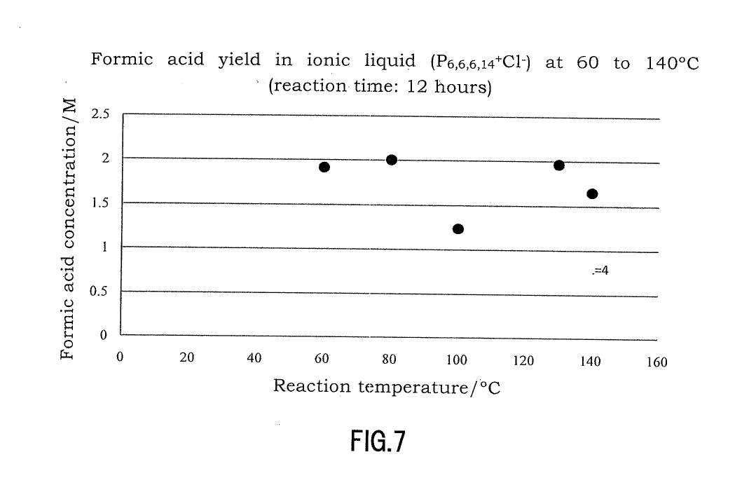

As a result, the formic acid yield (formic acid production) was found to be 24% (mol concentration: 1.65 M). FIG. 7 is a graph summarizing the relationship between the formic acid yield and the reaction temperature based on the abovementioned results. The graph indicates that formic acid can be synthesized even at a temperature as low as 60° C. Accordingly, it is decided that the formic acid synthesis temperature preferable for the ionic liquid is 60° C.

### Test Example 1

### Durability of Phosphonium Ion-Based Ionic Liquid

A mixture of formic acid and tri-n-hexyl-n-tetradecylphosphonium chloride in an amount of 0.29 mL (mole ratio: 0.6:1) was fed into a silica tube, which was then sealed, and reacted for 14 days in an electric furnace maintained at 250° C. Thereafter, the ionic liquid after the reaction was observed by NMR and the NMR spectrum obtained was compared with the NMR spectrum of the ionic liquid before the reaction to see presence/absence of a peak of any substance other than the products of the formic acid decomposition reaction to determine the durability of the ionic liquid.

The ¹H-NMR spectra were enlarged 200 times to see presence/absence of any minute peak, but no peak change was found other than those of formic acid and the products of the formic acid decomposition reaction to confirm that the ionic liquid was not deteriorated through the formic acid decomposition reaction at 250° C. for 14 days. It has been therefore decided that the ionic liquid is durable against repeated use to lead to significant cost reduction.

In addition, the same durability test was performed with 1-butyl-3-methylimidazolium bis(trifluoromethanesulfonyl)imide ((CF₃SO₂)₂N⁻) (purity: 99%) manufactured by Koei Chemical Co., Ltd. to find that this ionic liquid has less durability.

The result has revealed that tri-n-hexyl-n-tetradecylphosphonium chloride has extremely superior durability.

The abovementioned synthesis examples can be suitably applied to the apparatus due to the present invention.

According to the present invention, formic acid can be produced efficiently at low temperature using hydrogen and carbon dioxide as raw materials in the presence of an ionic liquid, in particular, an ionic liquid containing a phosphonium cation and in the absence of a catalyst.

What we claim is:

1. A formic acid producing apparatus comprising a closed formic acid synthesis reaction section to which an ionic liquid, hydrogen, and carbon dioxide are introduced externally, and in which formic acid is synthesized.

2. A formic acid producing apparatus according to claim 1, wherein the formic acid synthesis reaction section further comprises: a closed reactor having an outlet for sending out formic acid synthesized to an exterior; a heating means for heating at least the ionic liquid in the reactor; and a spreading part provided in the reactor for expanding a surface area of the ionic liquid in the reactor.

3. A formic acid producing apparatus according to claim 2, wherein the spreading part has a spreading wall; and a driving means for moving the spreading wall.

4. A formic acid producing apparatus according to claim 2, wherein the spreading part has a spreading wall; and a liquid circulation line for drawing the ionic liquid in the reactor and discharging the ionic liquid toward the spreading wall.

5. A formic acid producing apparatus according to claim 3, wherein the spreading wall has rough surfaces.

6. A method for producing formic acid comprising synthesizing formic acid from hydrogen and carbon dioxide in the presence of an ionic liquid using the formic acid producing apparatus according to claim 1.

7. A method for producing formic acid according to claim 6, wherein the ionic liquid is represented by the following general formula (1) 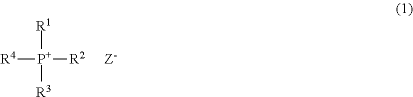
wherein
R¹, R², R³, and R⁴, the same or different, each represent an alkyl group that may be substituted with a halogen atom, or an aryl group or a heteroaryl group that may be substituted at the o-position or the p-position with 1 to 3 halogen atoms or a lower alkyl or alkoxy group, and
Z⁻ represents a counter anion for a phosphonium cation.

8. A method for producing formic acid according to claim 7, wherein in the general formula (1), R¹, R², R³, and R⁴ are an alkyl or aryl group having 1 to 18 carbon atoms.

9. A method for producing formic acid according to claim 7, wherein the phosphonium cation is a tetraphenylphosphonium salt, a tri-n-hexyl-n-tetradecylphosphonium salt or a tetrabutylphosphonium salt.

10. A method for producing formic acid according to claim 7, wherein the ionic liquid includes at least one of HCOO⁻, CF₃SO₃⁻, Cl⁻, NO₃⁻, and p-toluenesulfonate ion as counter anion.

11. A method for producing formic acid according to claim 6, wherein reaction temperature is from ambient to 200° C.
